(19) United States
(12) Patent Application Publication
(10) Pub. No.: US 2017/0105624 A1
(43) Pub. Date: Apr. 20, 2017

(54) Lower Extremity Visual Device

(71) Applicant: JONATHAN PAJOUH, Plano, TX (US)

(72) Inventor: JONATHAN PAJOUH, Plano, TX (US)

(21) Appl. No.: 14/884,374

(22) Filed: Oct. 15, 2015

(51) Int. Cl. A61B 5/00

(52) U.S. Cl. CPC A61B 5/0079

(57) ABSTRACT

The Lower Extremity Visual Device is a portable medical device that allows individuals as well as physicians the ability to view the lower extremity. With the use of mirrors and an attachable bracket for digital visual imaging devices such as an attachable camera, the patient can more readily identify abnormalities and aliments of the lower extremity of the body and track the progress of healing. The use of the mirrors and attachable bracket for digital imaging devices and recordable digital imaging devices allows individuals the ability to see portions of the lower extremity they were once unable to do so. This ability to quickly identify aliments and abnormalities early on is useful in preventive care.

### CROSS-REFERENCE TO RELATED APPLICATIONS

Not Applicable

### STATEMENT REGARDING FEDERALLY SPONSORED RESEARCH OR DEVELOPMENT Not Applicable

### REFERENCE TO SEQUENCE LISTING, A TABLE, OR A COMPUTER PROGRAM LISTING COMPACT DISC APPENDIX

Not Applicable

### BACKGROUND OF THE INVENTION

Lower extremity abnormalities and aliments are some of the most detrimental to day-to-day life of individuals. This is extremely true for individuals suffering from diabetes-induced neuropathy, severe obesity, and other physical impairments. Not being able to identify ulcers or injury early on leads to higher incidences of infection and further complications. Individuals become subject to expensive surgeries, life changing amputations, wound care and other treatments for something that if caught early on would be more effectively treated with less cost. Diabetic individuals especially those with neuropathy are at extreme risk. Individual who have lost feeling in their lower extremity are unable to detect an ulcer or wounds early on. Most of these individuals will not notice an ulcer or wound until it has become infected and severed damage has been done. Preventive care a vital aspect of day to day lives of individuals. Being able to view the lower extremity fully is a struggle most of these individuals face. The Lower Extremity Visual Device is a device that allows individuals to identify abnormalities early on by the use that they would be unable to see before.

### BRIEF SUMMARY OF INVENTION

The Lower Extremity Visual Device is a device that allows individuals to get a more detailed view of the lower extremity and view areas of the lower extremity that they were once unable to do so. This device allows the individual the ability to thoroughly view the lower extremity on a regular basis, and is time and cost effective. The advantage of the using this device helps increase an individual's ability to detect an abnormality for example an ulcer early on. This device plays a key role in preventive care because it gives these individuals the ability to identify abnormalities, record these abnormalities, and track progress. The goal of this device is to help detect abnormalities early so that the individuals are not subject to expensive and life altering treatments that could have easily been avoided.

### BRIEF DESCRIPTION OF THE SEVERAL VIEWS OF THE DRAWING

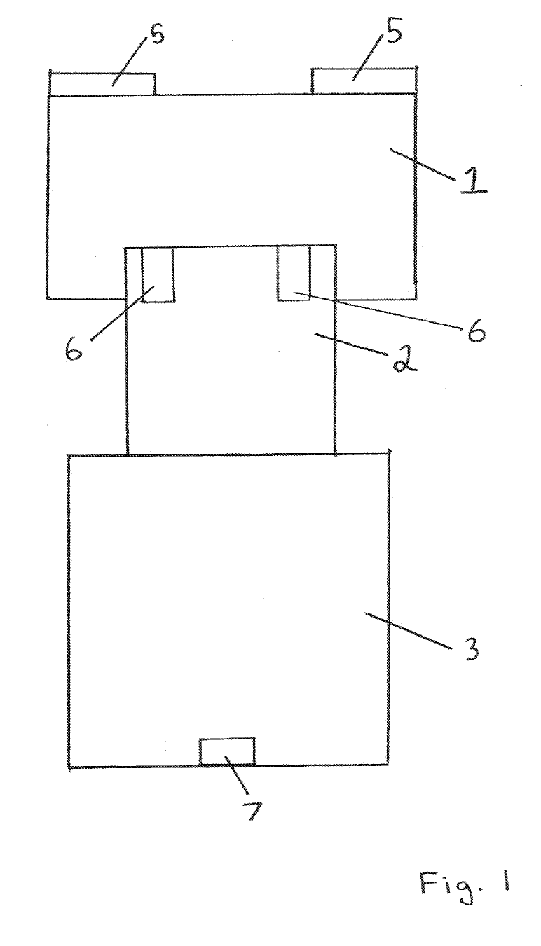

Depicted in FIG. 1. is a top view of the Lower Extremity Visual Device in its fully expanded form. This is the position that the device will be initially set to before the individual places it in their preferred position.

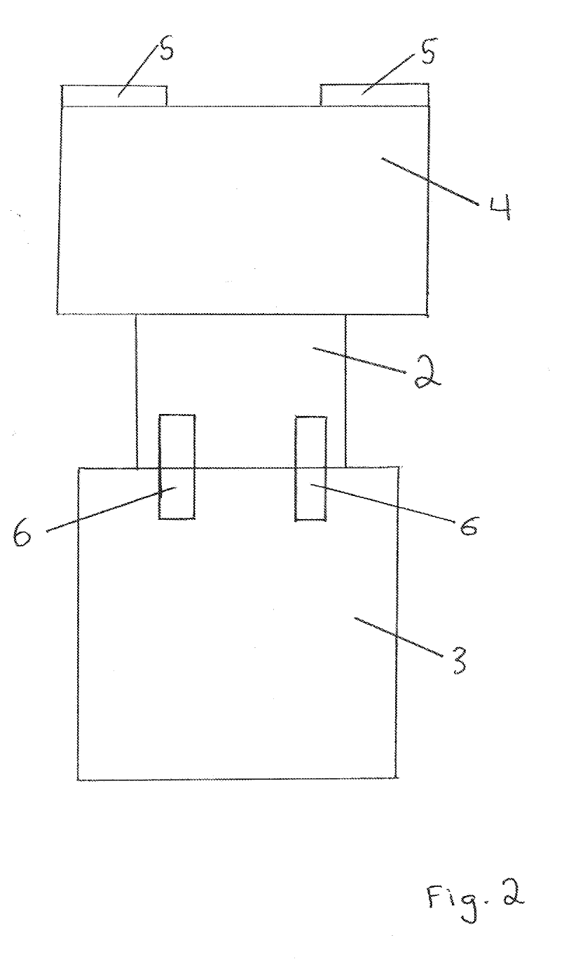

Depicted in FIG. 2. is a bottom view of the Lower Extremity Visual Device in its fully expanded form. This is the position that the device will be initially set to before the individual places it in their preferred position.

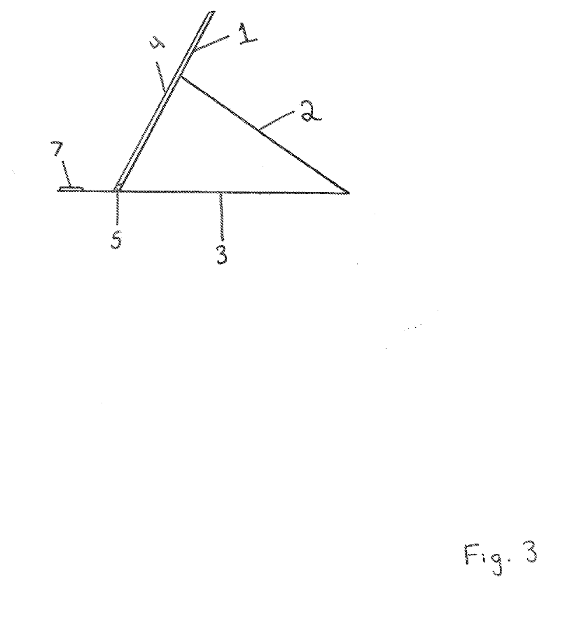

Depicted in FIG. 3. is a side view of the Lower Extremity Visual Device placed in functional form. This is one of the preferred positions that the individual may place the device in while trying to view the lower extremity. The angle of the component produced by items 1 and 4, can be adjusted to get a better viewing if needed by the individual.

### DETAILED DESCRIPTION OF THE INVENTION

To make the Lower Extremity Visual Device, the following items are required
Character 1:
   One pre cut piece of acrylic glass 0.08-inch thick, 14-inch width, 8-inch length
Character 2:
   One pre cut piece of acrylic glass 0.08-inch thick, 8-inch width, 8-inch length
Character 3:
   One pre cut piece of acrylic glass 0.08-inch thick, 12-inch width, 12-inch length
Character 4:
   One pre cut piece of mirrored glass 0.25-inch thick, 14-inch width, 8-inch length
Character 5:
   Two pieces of 4-inch in width, 1-inch length industrial door weather-strip
Character 6:
   Four pieces of flexible plastic brackets 0.01-inch thick, 2.88-inch width, 4-inch length
Character 7:
   One 0.08-inch thick, 2-inch width, 1-inch length mountings bracket
Character 8:
   8 oz of industrial strength clear glue

Instructions to create the device are:

1. Take the pre cut piece of acrylic glass 0.08-inch thick, 14-inch width, 8-inch length and apply 4 oz of industrial strength clear glue to the front of it. Once the glue has been applied, take the pre cut piece of mirrored glass 0.25-inch thick, 14-inch width, 8-inch length and place it on the pre cut piece of acrylic glass 0.08-inch thick, 14-inch width, 8-inch length in which the back is placed on top of the side which contains the glue. Allow these pieces to sit until the glue has fully dried. While those pieces are drying, take the pre cut piece of acrylic glass 0.08-inch thick, 8-inch width, 8-inch length and attach two pieces of flexible plastic bracket 0.01-inch thick, 2.88-inch width, 4-inch length on each side using the glue so that opposite sides contains two plastic bracket that are separated from each other by 2-inches and have 2-inches covering the acrylic glass. Attach using glue, one side containing the remaining 2-inches of the plastic bracket onto the bottom of the pre cut piece of acrylic glass 0.08-inch thick, 12-inch width, 12-inch length, so that the pre cut piece of acrylic glass 0.08-inch thick, 8-inch width, 8-inch length can fold onto the pre cut piece of acrylic glass 0.08-inch thick, 12-inch width, 12-inch length. Take the remaining two plastic brackets and attach it to the backside of the now dried mirror and glass component. Attach two pieces of 4-inch in width, 1-inch length industrial weather-strip to opposite sides of the mirror and glass component that are on the opposite side of the plastic brackets. Using 2 oz of industrial strength clear glue apply the 0.08-inch thick, 2-inch width, 1-inch length mountings bracket at the center edge of the pre cut piece of acrylic glass 0.08-inch thick, 12-inch width, 12-inch length. Attach the digital imaging device onto the 0.08-inch thick, 2-inch width, 1-inch length mountings bracket.

1. The Lower Extremity Visual Device is a portable visual device that can aid individuals in looking at the lower extremity.

2. The Lower Extremity Visual Device is a device that will be used by individuals who either require a better view of the lower extremity or are physically unable to view the lower extremity.
